(19) United States
(12) Patent Application Publication
(10) Pub. No.: US 2010/0098831 A1
(43) Pub. Date: Apr. 22, 2010

(54) NON INVASIVE DUAL BIOMETRIC IDENTIFICATION METHOD AND SYSTEM TO COLLECT AND SAFELY RETAIN FINGERPRINTS AND DNA FROM AN INDIVIDUAL

(76) Inventor: Flavio Gabriel Anderson, Arlington, VA (US)

Correspondence Address: FLASTER/GREENBERG P.C.;Four Penn Center, 1600 John F. Kennedy Boulevard, 2nd Floor, PHILADELPHIA, PA, 19103, US

(21) Appl. No.: 12/256,210

(22) Filed: Oct. 22, 2008

(51) Int. Cl. A61B 5/117

(52) U.S. Cl. 427/1; 118/31.5

(57) ABSTRACT

Non invasive method for collecting and retaining fingerprints and a DNA sample from an individual in a digit epithelial record comprising these steps: a) release the digit-epithelial record from its protection cover; b) lift the cover of the area with abrasive; c) rub the finger back and forth by pressing the finger; d) lower its cover; e) lift the cover of the area with graphite; f) rub the fingertip used in step c) by pressing the finger on the area with graphite; g) lower its cover; h) lift the cover of the area with adhesive; i) press the same fingertip used in step f) on the area with adhesive; J) lower its cover; and k)prepare the epithelial-digit record for later use. System to collect and safely retain digital impressions and a DNA sample from an individual by using the described method.

### BACKGROUND OF THE INVENTION

1. Field of the Invention

The invention refers to a non invasive Dual Biometric Identification Method and System to obtain and safely retain at least one fingerprint and a DNA sample to obtain a full DNA profile of an individual.

2. Description of Related Art

Since the discovery of the fingerprint as a personal identification element and, lately, the possibility of establishing with certainty the identity of a subject through the analysis of a sample of cells to determine his/her DNA, the registry of fingerprints and the capture of a sample to determine the DNA of an individual have seen an important evolution due to the necessity of counting with faithful and inviolable records.

The capture of fingerprints through its printing, registry or adhesion intents to be a preventive and deterrence method, a safety system for proving identities and a procedure to legitimize signatures and credentials submitted or endorsed in forms, documents, deeds, pages, etc., thus obtaining a database of the fingerprint and the recording of its valleys and ridges over the adhesive of the seal, as well as the particles of the released dead epithelial cells that remain within the seal allowing with some laboratory reagents and a tested protocol to establish the desired DNA profile, i.e., the genetic print of a person. Consequently, a confirmation of the identity of an individual through a double basis-graphic and genetic- is obtained with certainty.

This true Dual Biometric identification method is applicable to different procedures in order to obtain the authentication of assets, identities, signatures, credentials or documents such as: I.D., passports, driving licenses, etc., as well as forms or any kind of documentation, in order to discourage and/or prevent from an initial stage that any operation and/or civil transaction and/or direct or indirect business transaction, may become a fraud or illicit, an ideological false act or any kind of conspiracy, adulterating real identities or infringing the law, thus making easier the concealing of illegitimate acts.

There are countless prior devices or systems that have tried to achieve a safe authentication for the effective control of credit instruments and registered documents, such as U.S. Pat. Nos. 245,637 and 245,630.

The techniques that are currently known to legitimate assets and identities, signatures, credentials, forms or any kind of documentation, are not simple or easy to use, are expensive and lack real effectiveness.

Moreover, the already known and used techniques to print and register fingerprints of the thumb or the rest of the fingers of a person, e.g. through the use of inks, paints, sprays, etc., stain the fingers and a person must wash his/her hands to remove such ink, paint or spray. When printing over the paper, the ink usually drains and the procedure must be repeated.

Fingerprints obtained in such a way only give information to reproduce the fingerprint of a person, but do not register the thickness of the epithelial lines that constitute the fingerprint. Therefore, mistakes are made when measuring the distances between line and line, as the obtained drawing of the fingerprint does not reflect its depth.

U.S. Pat. No. 4,699,077 to Meadows, et al., granted on Oct. 13, 1987, refers to a compact fingerprinting system that includes an applicator comprising a sealed packet containing a porous pad impregnated with a solution of water-soluble, color-forming, marking compound, such as iron chloride having a controlled, optimum water content.

A print of a body part, such as a finger, is formed by opening the packet and wiping the distal portion of a finger on the pad, such as a folded towelette to form a non-visible, latent image pattern. The pattern is then transferred to a card which can contain an area impregnated with a developer for the marking compound. The developer reacts with the pattern to form dark, distinct, permanent images. The card and packet can be enclosed in an envelope to form a low profile, lightweight, mailable, self-administered fingerprinting system.

U.S. Pat. No. 5,143,551 granted on Sep. 1, 1992, refers to a single use fingerprint inking card to obtain fingerprints, which consists of a front sheet and a back sheet, the sheets being secured together in overlapping relationship, the front sheet being cut to define a plurality of openings with hinged re-closable panels covering the openings, an inking sheet having fingerprint ink on one side and secured between the front sheet and the back sheet with the side bearing the ink facing the openings, so that the panels can be individually opened to expose the fingerprint ink and thereafter closed to cover it. The nature of the inking sheet and the ink are such that the ink tends to cling to the inking sheet, not the facing sheet.

Patent application PCT/EP95/05015 published on Apr. 24, 1997 as WO 97/15032 (equivalent to the patent application Serial Nr. P960101195 published as AR000830A1 on Aug. 6, 1997) refers to a “System for the safe authentication and management of registered credit instruments”, which consists of a personalized document provided with a microprocessor to store a personal identification code (PIC) of the holder/bearer and information on biometric characteristics of the same, means for releasing said document to the holder, as well as different means for issuing registered credit instruments or documents, means for validating the credit instruments, all of which are provided with a device for acquiring at least one biometric characteristic of the holder/bearer. Said credit instruments comprise at least one security code univocally generated by the personal identification code—PIC similar to a cryptographic key; said information is associated to a mathematical expression of the data of the fingerprint of at least a finger of the holder/bearer of said document; the recognition for the issue of the credit instrument and the validation is obtained by comparing the information obtained from the direct reading of the finger of the holder/bearer and the application of the mathematical expression with the one stored in the document.

Said system uses the fingerprint of the individual to get a PIC with certain components and procedures; however, the system fails to provide effective results and is easily counterfeited by hackers when entered into the web. In addition, the system does not provide data about the depth of the finger pulp grooves and no information for determining the DNA is obtained.

U.S. Pat. No. 5,704,648 A1 granted on Jan. 6, 1998 (equivalent to the Argentine application Serial Nr. P960105399, granted as AR004352B1 on Jul. 15, 2005) discloses a “Removable replaceable re-adhesive label”, which is firmly adhered to a container, such as a first medical container. Thus, a portion of the label can be removed easily from that container and adhered once again to a second medical container or to a conservation material from a medical archive where other information is stored, while the portion of the label remaining over the container or original device allows to identify its original content, being possible to eliminate such device and/or its content. The label is made from an elongated sheet having a front surface, a rear surface, a first end, a second end, a top edge and a bottom edge. The sheet includes a removable portion and a remaining portion. Both the removable portion and the remaining portion have identifying indicia printed thereon. The rear surface has an adhesive coating applied thereto. An adhesive reducing coating is applied to the adhesive coating adjacent to the first end of the elongated sheet, which allows that a portion of the sheet be removable. Preferably, said sheet is made from a uniaxially oriented material. Even though this label overcomes certain technical deficiencies, the same provides no identification through a double code and is unable to recognize unmistakably an individual associated to the container. Also, it is not possible to identify an individual through its fingerprint or DNA.

Finally, U.S. Pat. No. 6,659,038 B2 published on Dec. 9, 2003 (equivalent to patent AR033954B1 granted on Nov. 29, 2005, Serial Nr. P000105051), refers to a safety identification device for collecting fingerprints and DNA-related material from an individual. The device is used to obtain the fingerprints of an individual and a sample of epithelial cells from the finger, which are used to characterize a DNA with some laboratory reagents. The purpose of the invention is to be a preventive and deterrence procedure, a procedure for legitimizing signatures in credentials, ID cards, forms, banking documents, deeds, credit card coupons, multi-code cards, web pages and any kind of direct or indirect document, etc. The device comprises three components. The device is rectangular, with long sides having 55-65 mm and short sides having 25-35 mm.

The first component is a base layer comprising a base sheet made of high resistant high density glassine super-calendered kraft paper having an inner silicone treated smooth semi-glossy face with a thickness of 65-75μ, with high resistance of 0.59-7.87 kg/cm and a density of 76-85 g/m², with a flap which sticks out about 3 mm in isoplanar shape. The second component is located at the center of the arrangement and comprises a double sided A-108 support material comprising a non-thermally sealable polypropylene film having a tensile strength of 1400 kg/square inch, a weight of approx. 11 kg/square meter, a thickness of approx. 12.5. mu.) and crystal color. Both faces of the intermediate layer are provided with an adhesive comprising an aqueous dispersed acrylate polymer modified or in acetate, with good initial adhesiveness, and having high cohesive properties at 24 h, applicable over any surface. The third component or layer comprises a high strength, high density glassine-type super-calendered Kraft paper having a generally crystalline-white color. The inner face allows to be printed while the outer face is non-adhesive. The inconvenience of this device is that the adhered cells fail to secure the identification of the individual, as the sample obtained does not permit a full profile identification.

Consequently, in order to achieve a multiple record of scannable fingerprints and DNA samples that may be used to confirm the identity of an individual with almost 100% effectiveness, it is necessary to implement a method and an easy-to-use system allowing said purpose.

### BRIEF SUMMARY OF THE INVENTION

It is therefore an object of the present invention to provide a non invasive dual biometric identification method to collect and safely retain fingerprints and a DNA sample of an individual with a digit-epithelial (fingerprinting and epithelial cell sample) record comprising three areas.

One area with graphite, one area with an abrasive surface and one area with adhesive for fingerprint collection. All the areas are fixed to a laminar cellulose support; this method comprises the following steps:

a) release the digit-epithelial record from its protection cover;

b) lift the cover of the area with abrasive;

c) rub the finger back and forth by pressing the finger on the area with abrasive;

d) lower the cover of the abrasive area to retain the collected cellular material;

e) lift the cover of the area with graphite;

f) rub the fingertip used in step c) by pressing the finger on the area with graphite;

g) lower the cover of the area with graphite;

h) lift the cover of the area with adhesive;

i) press the same fingertip used in step f) on the area with adhesive, in a normal way and without displacement.

j) lower the cover of the area with adhesive and collect the fingerprint to retain the fingerprint obtained in step i);

k) prepare the epithelial-digit record for later use.

In order to obtain a representative sample of epithelial cells, the finger is rubbed preferably on its side zone, pressing firmly on the abrasive surface, during at least eight cycles, where one cycle is equal to one “back and one forth” movement without lifting the finger, maintaining it always in contact with the abrasive element.

In order to obtain the fingerprint, the finger is rubbed on its central zone making pressure over the graphite zone, during a at least five cycles, where one cycle is equal to one “back and one forth” movement without lifting the finger, maintaining it always in contact with the graphite element.

In addition, such method may comprise the following steps:

l) soak a swab into alcohol;

m) lift the cover of the area with abrasive in order to disclose the sample of epithelial cells previously obtained;

n) lift the epithelial cells by using a swab, soaked in alcohol, lifting at least part of the epithelial cells that are in the abrasive area;

o) Place the swab with adhered epithelial cells inside a device for processing and determination of DNA profile; and

p) lower the cover of the area with abrasive to preserve the rest of the epithelial cell material collected.

Preferably, the finger used to obtain the epithelial sample and the fingerprint is the thumb.

The aforementioned method also comprises the steps of lifting the fingerprint according to the traditional methods used by the scientific/forensic police and using a powder thinner than talcum and a Marten-hair brush.

An accuracy of 99.9% in the determination of a full DNA profile of an individual is reached by taking a sample of epithelial material from the abrasive surface using a cotton swab soaked in ethylic alcohol.

Another purpose of this invention is a system to collect and safely retain digital impressions and a DNA sample from an individual by using the method described above, which comprises:

a laminar cellulose support, substantially rectangular with three substantially defined and parallel disposed areas, which comprises:
one first area that comprises a tape bearing graphite in solid suspension;
a second area that comprises an abrasive surface; and
a third area that comprises a surface with a superficial adhesive to collect fingerprints;
the first, second and third area s comprise a flexible and transparent plastic removable cover, fixed laterally as a hinge allowing said cover to be lifted in order to expose each area to the user and to be lowered to retain the fingerprint and the sample containing information to determine a full DNA profile of the person, who stamped his finger in the third area.

The first area consists of three superimposed components which present a flat rectangular ornamental aspect. Its components are: a plastic material base with adhesive in both sides, fixed to the support base in one of its sides as a first component, a second component which is a graphite supporting tape in solid suspension bearing said graphite is fixed to the opposite side; the third component is a transparent flexible plastic material film fixed laterally as a hinge with its interior face covered with silicone.

The second area consists of three superimposed components which present a flat rectangular ornamental aspect. Its components are: a plastic material base with adhesive in both sides fixed to the base of the support in one of its side as a first component, an abrasive surface is fixed to the opposite side as a second component, and the third component is a transparent flexible plastic material film fixed laterally as a hinge with its interior face covered with silicone.

The third area consists of two superimposed components which present a flat rectangular ornamental aspect. Its components are: a plastic material base with adhesive in both sides fixed to the base of the support in one of its side as a first component, and over the adhesive layer on the opposite side, the second component which is a transparent flexible plastic material film fixed laterally as a hinge with its interior face covered with silicone.

The back side of the laminar cellulose support comprises a label with printings. Most preferably, such printings include inscriptions, trademarks, logos, details that enable the provision of instructions for use, batch numbers, and combinations thereof.

In particular, the area with graphite is graphite powder in solid suspension. The graphite powder can be in the shape of charcoal activated in powder, soot, fine graphite powder or super fine powder of any charcoal in general in solid suspension. Said, solid suspension graphite may comprise: silicon, polyethylen glycol, high molecular weight fat, synthetic resins, synthetic polymers, or mixes thereof, placed on a cellulose support like paper, in such a way that the adherence of the graphite to the support allows its removal when the fingertips are pressed on the surface.

On the other hand, the area with abrasive is composed of an abrasive surface formed by abrasive paper comprising an abrasive material adhered to a cellulose support, fabric or made of fiber, forming a resinous matrix with an appropriate adhesive.

The cellulose support is Kraft paper, the abrasive material is selected from: Silicon carbide, aluminum oxide, granate, talcum, or its mixes thereof, and the adhesive is acrylic type.

The area with abrasive is an abrasive film with a grain size distribution between 100 and 600, preferably between 180 and 320, and more preferably among 240.

The adhesive that constitutes the aforementioned system is a pressure sensitive adhesive (PSA). The PSA is selected from the group of: rubber-based adhesives, polymer adhesives or solvent-based acrylic copolymers, and polymer adhesives or acrylic copolymer in emulsion.

The covers of the first, second and third areas are from a selected polymer consisting in: Polyethylene (PE), polypropylene (PP), polystyrene (PS), Polyvinyl chloride (PVC) and polyurethane (PU). Specifically, the polymer has a low, medium or high density and has a white, transparent or crystal-like appearance.

Having described the Dual Biometric Identification method and the system to collect and retain safely fingerprints and a DNA sample from an individual in a digit epithelial record and the formation of the system, it can be concluded how useful and practical its use can be in any time.

Making clear any difference with any already existing method and/or system, and considering each part of the invention and its functions and features, this method is absolutely original for its legal registration.

Other purposes, aspects and additional advantages of this invention will appear through its subsequent detail description and based on a clear example.

In order to obtain a clear explanation of the nature of this invention, as well as the form in which the same should be put into practice, attached hereto is an example with illustrative figures.

It must be pointed out that such practice is a mere example. The same is just illustrative and does not reflect the complete scope and application of the invention per sé. Same reference numbers appearing in the enclosed figures relate to similar parts.

### BRIEF DESCRIPTION OF THE SEVERAL VIEWS OF THE DRAWING(S)

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 shows a perspective view of the system of an identification device according to the invention by which fingerprints and DNA are obtained and preserved;

FIG. 2 shows a cross sectional view of the system of FIG. 1;

FIG. 3 shows Step “c” of the method to obtain and retain fingerprints and a DNA sample in an epithelial digit record according to the invention. The arrows show the finger movements in the abrasive surface.

FIG. 4 shows Step “f” of the method to obtain and retain fingerprints and a DNA sample in an epithelial digit record according to the invention. The arrows show the finger movements in the graphite surface.

FIG. 5 shows Step “i” of the method to obtain and retain fingerprints and a DNA sample in an epithelial digit record according to the invention. The arrows show the finger pressure on the adhesive surface.

FIG. 6 shows Step “j” of the method to obtain and retain fingerprints and a DNA sample in an epithelial digit record according to the invention.

FIG. 7 shows Step “n” of the method to obtain and retain fingerprints and a DNA sample in an epithelial digit record according to the invention. The arrows show the cotton swab (soaked in alcohol) movements on the area with adhesive.

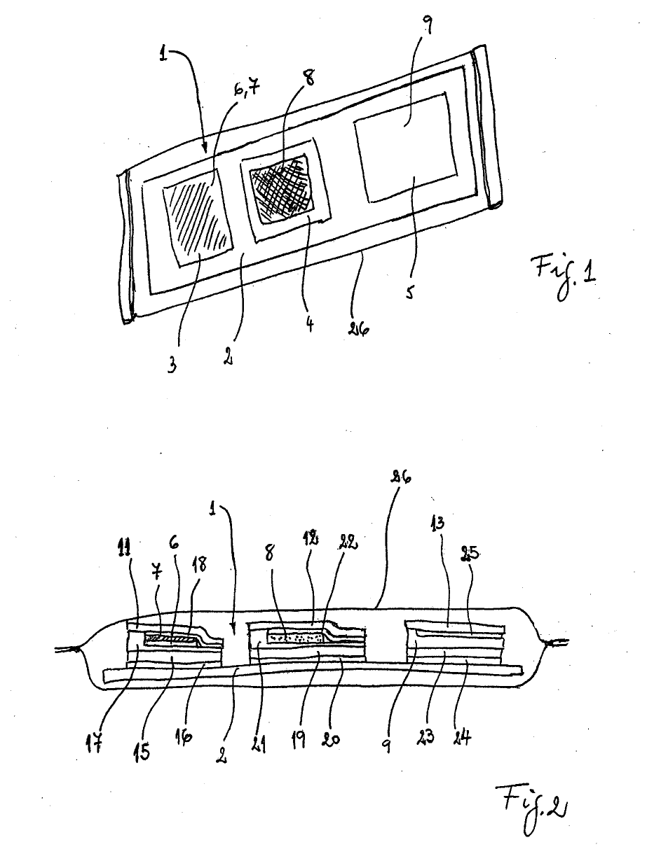

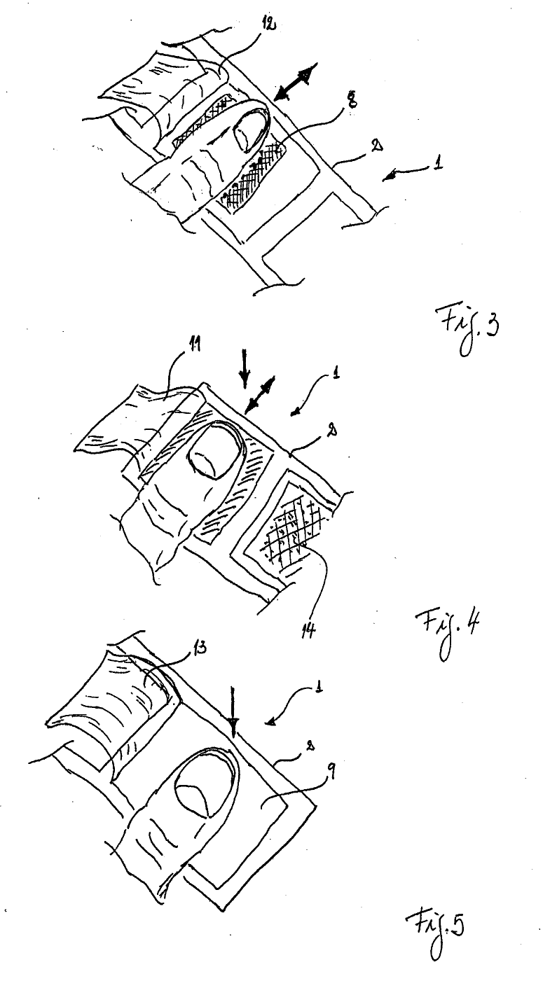

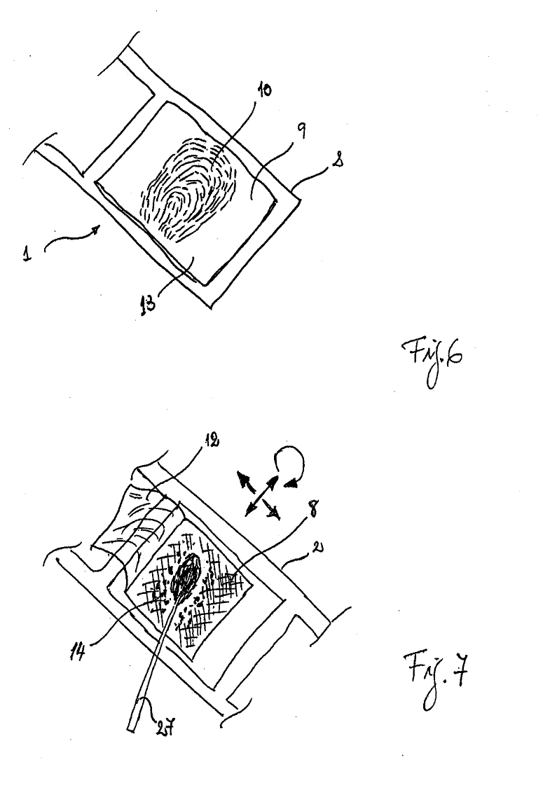

### DETAILED DESCRIPTION OF THE INVENTION

It is known that a fingerprint is a roughness with an arbitrary shape formed by the skin covering the fingertip. Fingerprints are made of grooves forming ridges and valleys. The ridges are the papillary ridges and the valleys are the inter-papillary areas. The ridges hold the glands that produce sweat, which has grease that slides onto the valleys, and accumulates there.

When grabbing an object by the hands, the fingers leave marks or fingerprints that cannot be perceived at first sight. The reason for this is that when an object is grabbed, tiny particles of sweat, grease and other substances, which are normally kept in the valleys of fingerprints, are left.

These minuscule residues leave a latent image of the fingers printed in the form of fingerprints, as a negative of the valleys of said prints.

Normally, when the surface that contains the fingerprint is white or of bright colors, the latent image may be revealed by using extremely fine activated charcoal powder, while for dark surfaces, talcum is used. Both activated charcoal and talcum particles adhere to the above referenced invisible particles by adsorption, marking the outlines of the latent fingerprint with black or white color, visible as it contrasts with the color of the object where the print was left.

The scientific/forensic police use more technical instruments and specific powders for different types of surfaces.

These marks or fingerprints comprise patterns and indicators that serve to identify a person, but in order to have a reliable record of the identity of an individual it is convenient to have additional data for confirmation.

For a long time, taking fingerprints of all the fingers from both hands to have the entire set of individual marks, was the method used. However, presently technology developments have made it possible for this verification to be performed by using a person's DNA analysis as the final and absolute confirmation.

The purpose is to get a sample to effectively perform a DNA test without using invasive or complex methods and to be able to easily perform a cross-identification using at least one fingerprint. Hence, safe files could be made in a much faster and more effective and efficient manner.

Therefore, considering the above, the subject of this invention is a non invasive Dual Biometric Identification Method and System to collect and retain fingerprints and a DNA sample of an individual in a fingertip-epithelial record, comprising three areas: one with graphite, one with an abrasive surface, and one with adhesive to take fingerprint impressions; where all the areas are fixed to a laminar cellulose support. Such method comprises the following steps:

a) release the digit-epithelial record from its protection cover;

b) lift the cover of the area with abrasive;

c) rub the finger back and forth by pressing the finger on the abrasive area;

d) lower the cover of the abrasive area to preserve the collected cellular material;

e) lift the cover of the area with graphite;

f) rub the fingertip used in step c) by pressing the finger on the area with graphite;

g) lower the cover of the area with graphite;

h) lift the cover of the area with adhesive;

i) press the same fingertip used in step f) on the area with adhesive, in a normal way and without displacement.

j) lower the cover of the area with adhesive and collect the fingerprint to preserve the fingerprint obtained in step i);

k) prepare the epithelial-digit record for later use.

Preferably, to obtain a representative sample of epithelial cells, the finger is rubbed, preferably on its side zone, pressing firmly on the abrasive area, during a at least eight cycles, where one cycle is equal to one back and one forth movement without lifting the finger, keeping it always in contact with the abrasive.

In order to take a fingerprint impression of one finger, this is rubbed preferably from its center pressing the graphite area, for at least five cycles, where one cycle equals one movement back and one forward without lifting the fingertip, keeping it always in contact with the graphite.

In addition, such method may comprise the following steps:

l) soak a swab into alcohol;

m) lift the cover of the area with abrasive in order to disclose the sample of epithelial cells previously obtained;

n) lift the epithelial cells by using a swab, soaked in alcohol with adhesive, at least part of the epithelial cells that are in the abrasive area;

o) Place the swab with adhered epithelial cells inside a device for processing and determination of DNA profile; and

p) lower the cover of the area with abrasive to preserve the rest of the epithelial cell material collected.

Preferably, the finger used to obtain the epithelium sample and the fingerprint is the thumb.

Moreover, the area with graphite is formed by graphite powder in solid suspension, where such graphite powder may be in the shape of charcoal activated powder, soot, fine graphite powder or super fine powder of any charcoal in general, in solid suspension.

Said solid graphite suspension may comprise silicone, polyethylene glycol, high molecular weight fat, synthetic resins, synthetics polymers, or mixes thereof, placed on a cellulose support like paper in such a way that the adherence of the graphite to the device allows its removal when the fingertips are pressed on this surface.

On the other hand, the abrasive area is composed of an abrasive surface formed by abrasive paper comprising an abrasive material adhered to a cellulose support, fabric or made of fiber, forming a resinous matrix with an appropriate adhesive. Preferably, the cellulose support is Kraft paper, the abrasive material is selected among silicon carbide, aluminum oxide, granate, talcum, and mixes thereof, and the adhesive is of acrylic type.

The abrasive area is preferably an abrasive film of grain size distribution comprised between 100 and 600, preferably between 180 and 320, and more preferably around 240.

In particular, the adhesive of step h) or of step l) is a Pressure Sensitive Adhesive (PSA).

Preferably, the PSA is selected from the group of: rubber-based adhesives, polymer adhesives or solvent-based acrylic copolymers, and polymer adhesives or acrylic copolymer in emulsion.

Preferably, the covers of the first, second and third areas are from a selected polymer consisting in: Polyethylene (PE), polypropylene (PP), polystyrene (PS), Polyvinyl chloride (PVC) and polyurethane (PU). Specifically, the polymer has a low, medium or high density and has a white, transparent or crystal-like appearance.

More preferably, the covers of first, second and third areas should be transparent.

One of the purposes of the covers of the first, second and third areas is to preserve and maintain the fingerprint and DNA sample obtained free of contamination.

The method described above may include the additional steps of taking the fingerprint with an optical scanner and/or optical character reader through the transparent cover of the relevant area, and saving the information in a digital record.

On the other hand, the method may comprise the additional steps of lifting the fingerprint according to the traditional methods used by the scientific/forensic police and using a powder thinner than talcum and a Marten-hair brush.

In order to take the sample of epithelial cells left on the abrasive surface a swab is used, preferably a cotton swab soaked in ethylic alcohol.

The proposed method makes it possible to get a sample of the cells that enable the determination of the DNA of an individual for the sole purpose of his/her full profile identification, with a 99.9% of certainty.

With the result of the DNA analysis a complete identification profile according to the FBI/NIST standards can be obtained from the DNA sample taken.

Another purpose of this invention is a system (1) to collect and safely retain fingerprint impressions and a DNA sample of a person through the method of any of the prior claims, comprising:

A support (2) of laminar cellulose support mainly rectangular on which, three areas are displayed (3, 4, 5), mainly rectangular, defined and parallel to one another on said base (2), comprising:

a first area (3) that consists of a tape (6) that supports graphite (7) in a solid suspension;

a second area (4) that comprises an abrasive surface (8); and

a third area (5) that comprises a cover of adhesive (9) to deposit a fingerprint (10); and

where the first (3), second (4) and third (5) areas comprise a removable cover (11, 12, 13) of flexible and transparent plastic material fixed on the side as a hinge enabling said covers (11, 12, 13) to be lifted to expose each area (3, 4 and 5) to a user and to be lowered to preserve the fingerprint (10) and the sample (14) with enough information to find out in a laboratory the DNA of the individual who stamped his thumb on the third area (5).

Preferably, the first area (3) consists of three superimposed components, which have an ornamental rectangular flat aspect, where said components are: a base (15) of plastic material with adhesive (17, 16) on both sides fixed to the support base (2) on one of its sides as a first component. On the opposite side a second component, a graphite support tape (6) in solid suspension comprising said graphite (7) is fixed, and the third component is a film (11) of flexible plastic and transparent material fixed on its side as a hinge, whose inner side is covered with silicone (18).

Preferably, the second area (4) consists of three superimposed components, which present an ornamental rectangular flat aspect, where said components are: a base (19) of plastic material with adhesive (20, 21) on both sides fixed to the support base (2) by one of its sides as a first component. On the opposite side an abrasive surface is fixed (8) as second component, and the third component is a film (12) of plastic flexible and transparent material fixed on its side as a hinge, whose inner side is covered with silicone (22).

Preferably, the third area (5) consists of two superimposed components, which have an ornamental rectangular flat aspect, where the components are: a base (23) of plastic material with adhesive (24, 9) on both sides fixed to the support base (2) by one of its sides as a first component, and on the adhesive coat (9) of the opposite side the second component, a film (13) of flexible and transparent plastic material, is displayed, fixed on its side as a hinge, whose inner side is covered by silicone (25).

Preferably, the system (1) should be preserved by a protective wrapper (26) or an ad hoc bag, e.g. made of cellophane or of a plastic material, as polyethylene (PE).

In particular, the system (1) to obtain and safely preserve fingerprints and a DNA sample of an individual comprises:

a support (2) of laminar cellulose material, mainly rectangular on which three mainly rectangular areas (3, 4, 5) defined and displayed in parallel, comprising:

a first area (3) formed by three superimposed components, with an ornamental rectangular flat aspect, where said components are: a base (15) of plastic material with adhesive (16, 17) on both sides fixed to the support base material (2) by one of its sides as a first component; on the opposite side a second component is fixed, which is a support graphite tape (6) in solid suspension of graphite including said graphite (7), and the third component is a film (11) of flexible plastic and transparent material fixed laterally on one side as a hinge whose inner side is covered by silicone (18);

a second area (4) formed by three superimposed components, with an ornamental rectangular flat aspect, where said components are: a base (19) of plastic material with adhesive (20, 21) on both sides, fixed to the support base (2) by one of its sides as a first component, on the opposite side an abrasive surface is fixed (8) as a second component, and the third component is a film (12) of plastic flexible and transparent material laterally fixed as a hinge, whose inner side is covered with silicone (22); and

a third area (5) formed by two superimposed components, with an ornamental rectangular flat aspect, where these are: a base (23) of plastic material with adhesive (24, 9) on both sides, fixed to the base material (2) by one of its sides as a first component, and on the adhesive layer (9) of the opposite side a second component, which is a film (13) of plastic flexible and transparent material laterally fixed as a hinge whose inner side is covered with silicone (25), is set out;

where the first component of the first (3), second (4) and third (5) areas are a removable cover (11, 12, 13) of plastic flexible and transparent material laterally fixed as a hinge, enabling said covers (11, 13) to be lifted to expose each area (3, 4, 5) to a user, and to be lowered to preserve the fingerprint (10) and the obtained sample (14) of epithelial cells which will permit the determination in a Laboratory of a full DNA profile of the individual, who has stamped his finger on the third area (5).

Preferably, the back side of said the laminar cellulose support (2) of the system carries an adhered label with printings or bears printings on it.

More preferably, the printings are selected from the group of inscriptions, trademarks, logos, details that enable the provision of instructions for use, batch number, and combinations thereof.

Preferably, the area (3) with graphite (7) is graphite powder in solid suspension. In particular, the graphite powder may be in the form of activated charcoal powder, soot, fine graphite powder, or super fine powder of any charcoal in general, in solid suspension. On the other hand, the solid graphite suspension (7) may comprise silicone, polyethylene glycol, high-molecular weight fat, synthetic resins, synthetic polymers, or mixes thereof, being deposited on a cellulose support or tape (6) such as paper, so that the graphite adherence (7) to the support be such that it enables its removal when applying the fingertips on this surface.

Preferably, the area (4) with abrasive (8) is composed of an abrasive paper formed by an abrasive material (8) adhered to a cellulose support, fabric or fiber support, forming a resinous matrix with a suitable adhesive.

In particular, the cellulose support is Kraft paper, the abrasive material is selected among silicon carbide, aluminum oxide, granate, talcum and mixes thereof, and the adhesive is of acrylic type.

Preferably, the area (4) with abrasive (8) is an abrasive film of grain size distribution between 100 and 600, preferably between 180 and 320, and more preferably around 240.

In order to collect the sample of epithelial cells left on the abrasive surface a swab (27) is used, preferably a cotton swab (27) soaked in ethylic alcohol.

Preferably, the adhesive (9) of the third (5) area is a pressure sensitive adhesive (PSA). More preferably, the PSA is selected from the group consisting of a rubber-base adhesive, polymer adhesive or acrylic copolymer in a solvent base, and polymer or copolymer adhesive in emulsion.

In particular, the layers (11, 12, 13) of the first (3), second (4) and third (5) areas are of a polymer selected from the group consisting of: polyethylene (PE), polypropylene (PP), polystyrene (PS), polyvinyl chloride (PVC), and polyurethane (PU). Preferably, the polymer is of a low, medium or high density, and has a white transparent or crystal appearance.

Preferably, the layers (11, 12, 13) of the first (3), second (4) and third (5) area are transparent.

One of the purposes of the layers (11, 12, 13) of the first (3), second (4) and third (5) area is to preserve and maintain the fingerprint and DNA sample obtained free of contamination.

It is understood that the foregoing description is only for clarifying purposes and that it may be subject to modifications and variations without deviating from the spirit and scope of this invention.

Although the invention in these technical specifications was described as a hands-on approach example, the bases of the invention itself are clear and the terms of the claims are established herein below, as well.

It should be noted that as of this date, the applicant's best method to implement the above invention is the conventional method for its manufacture.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A non invasive Dual Biometric Identification Method to collect and safely retain fingerprints and a DNA sample to obtain a full DNA profile of an individual in a digit epithelial record comprising three areas: one area with graphite, one area with an abrasive surface and one area with adhesive for fingerprint collecting, wherein the areas are fixed to a laminar cellulose support, compromising:
a) releasing the digit-epithelial record from its protection cover;
b) lifting the cover of the area with abrasive;
c) rubbing the finger back and forth by pressing the finger on the area with abrasive;
d) lowering the cover of the area with abrasive to preserve the collected cellular material;
e) lifting the cover of the area with graphite;
f) rubbing the fingertip used in step c) by pressing the finger on the area with graphite;
g) lowering the cover of the area with graphite;
h) lifting the cover of the area with adhesive;
i) pressing the same fingertip used in step f) on the area with adhesive, in a normal way and without displacement.
j) lowering the cover of the area with adhesive and collect the fingerprint to preserve the fingerprint obtained in step i);
k) preparing the epithelial-digit record for later use.

2. The method of claim 1, wherein in order to obtain a sample of epithelial cells in step c), the finger is rubbed on its side, pressing firmly on the abrasive area, during at least eight cycles, wherein each cycle equals one “back and forth” movement without lifting the finger and maintaining it in contact to the abrasive.

3. The method of claim 1, wherein in order to collect the fingerprint in step f), the finger is rubbed on its central zone pressing firmly on the graphite are, during at least five cycles, wherein each cycle equals one “back and forth” movement without lifting the fingertip and maintaining it in contact to the graphite.

4. The method of claim 1, which further comprises the following steps:
l) soaking a swab into alcohol;
m) lifting the cover of the area with abrasive in order to disclose the sample of epithelial cells previously obtained;
n) lifting the epithelial cells by using a swab, soaked in alcohol with adhesive, at least part of the epithelial cells that are in the abrasive area;
o) placing the swab with adhered epithelial cells inside a device for processing and determination of DNA profile; and
p) lowering the cover of the area with abrasive to preserve the rest of the epithelial cell material collected.

5. The method of claim 1, wherein the finger used to collect the epithelial sample and fingerprint is the thumb.

6. The method of claim 1, wherein the graphite zone is graphite powder in solid suspension.

7. The method of claim 6, wherein the graphite powder is a form chosen from activated charcoal powder, soot, fine graphite powder or super fine graphite powder of any charcoal in solid suspension.

8. The method of claim 7, wherein the graphite solid suspension comprises a material chosen from silicone, polyethylen glycol, high molecular weight fat, synthetic resins, synthetic polymers, or mixtures thereof, placed on a cellulose support such as paper, in such a way that the adherence of the graphite to the device allows its removal when the fingertips are pressed on the surface.

9. The method of claim 1, wherein the area with abrasive is formed by an abrasive paper comprising an abrasive material adhered to a cellulose support, fabric or made of fiber, forming a resinous matrix with an appropriate adhesive.

10. The method of claim 9, wherein the cellulose support is Kraft paper.

11. The method of claim 9, wherein the abrasive material is chosen from: silicon carbide, aluminum oxide, granate, talcum, and mixes thereof.

12. The method of claim 9, wherein the adhesive comprises an acrylic.

13. The method of any of claim 9, wherein the abrasive paper is an abrasive film with a grain size distribution between 100 and 600.

14. The method of claim 1, wherein the adhesive of step h) is a pressure sensitive adhesive (PSA).

15. The method of claim 14, wherein the PSA is chosen from rubber-based adhesive, polymer adhesive or copolymer acrylic adhesive in solvent base, and polymer adhesives or copolymer acrylic in emulsion.

16. The method of claim 1, wherein the first, second and third areas each have a cover comprising a polymer chosen from polyethylene (PE), polypropylene (PP), polystyrene (PS), Polyvinyl chloride (PVC) and polyurethane (PU).

17. The method of claim 16, wherein the polymer has a low, medium or high density and a white, transparent or crystal-like appearance.

18. The method of claim 16, wherein the covers of the first, second and third areas are transparent.

19. The method of claim 16, wherein one of the purposes of the covers of the first, second and third areas is to preserve and maintain the fingerprint and DNA sample obtained free of contamination.

20. The method of claim 18, wherein comprises the additional step of scanning with an optical scanner and/or optical character reader the fingerprint through the transparent cover from the corresponding area and saving the information in a digital record.

21. The method of claim 1, wherein comprises the additional step of lifting the fingerprint according to the traditional methods used by the scientific/forensic police using a powder thinner than talcum and a Marten-hair brush.

22. The method of claim 4, wherein the swab is a cotton swab.

23. The method of claim 4, wherein the alcohol is ethylic alcohol.

24. The method of claim 4, wherein an accuracy of 99.9% in the determination of the DNA of an individual is reached.

25. The method of claim 24, wherein with the analysis of the DNA sample, a full DNA profile of identification, according to standards of the FBI/NIST, is reached.

26. A system to collect and safely preserve digital impressions and a DNA sample of an individual comprising a support of laminar cellulose material mainly rectangular in which three areas are displayed, defined and parallel to one another in said base, comprising:
one first area that consists of a tape that supports graphite in solid suspension;
a second area that comprises an abrasive surface; and
a third area that comprises a surface with a superficial adhesive to collect a fingerprint;
wherein, the first, second and third areas comprise a removable cover of flexible and transparent plastic material, fixed on the side as a hinge enabling said covers to be lifted in order to expose each area to a user and to be lowered to preserve the fingerprint and the sample containing enough information to determine the DNA of a person, who stamped its finger in the third area.

27. The system of claim 26, wherein the first zone is composed of three superimposed components, which present a flat rectangular ornamental aspect, wherein the first component comprises a base of plastic material with adhesive on both sides, fixed to the support base in one of its sides, a second component, comprising a graphite support tape in solid suspension comprising said graphite is fixed, and the third component comprising film of flexible plastic and transparent material fixed on its side as a hinge, whose inner side is covered with silicone.

28. The system of claim 26, wherein the second area is composed of three superimposed components, which present an ornamental flat rectangular aspect, wherein the first component comprises a base of plastic material with adhesive on both sides fixed to the support base by one of its sides as a first component, the second component comprises an abrasive surface is fixed, and the third component comprises film of flexible plastic and transparent material fixed on its side as a hinge whose inner side is covered with silicone.

29. The system of claim 26, wherein the third area is composed of two superimposed components, which present an ornamental flat rectangular aspect, wherein the components are: a base of plastic material with adhesive in both sides fixed to the support base by one of its side as a first component, and on the adhesive coat of the opposite side is the second component, a film of flexible transparent plastic material is displayed fixed on its side as a hinge, whose inner side is covered with silicone.

30. The system of any of the claim 26, wherein the system is preserved in a protective wrapper or a bag.

31. A system to obtain and safely preserve fingerprints and a DNA sample of an individual comprising:
a support of laminar cellulose mainly rectangular in which three areas are displayed, defined and parallel to one another in said base, comprising:
a first zone composed of three superimposed components, which present a flat rectangular ornamental aspect. The components of said areas are: a base of plastic material with adhesive on both sides, fixed to the support base in one of its sides as a first component. On the opposite a second component, a graphite support tape in solid suspension comprising said graphite is fixed, and the third component is a film of flexible plastic and transparent material fixed on its side as a hinge, whose inner side is covered with silicone;
a second area composed of three superimposed components, which present an ornamental flat rectangular aspect, wherein the components are: a base of plastic material with adhesive on both sides fixed to the support base by one of its sides as a first component. On the opposite side a second component, an abrasive surface is fixed, and the third component is a film of flexible plastic and transparent material fixed on its side as a hinge whose inner side is covered with silicone; and
a third area composed of two superimposed components, which present an ornamental flat rectangular aspect, wherein the components are: a base of plastic material with adhesive in both sides fixed to the support base by one of its side as a first component, and on the adhesive coat of the opposite side is the second component, a film of flexible transparent plastic material is displayed fixed on its side as a hinge, whose inner side is covered with silicone;
wherein, the first, second and third areas comprise a removable cover of flexible and transparent plastic material, fixed on the side as a hinge enabling said covers to be lifted in order to expose each area to a user and to be lowered to preserve the fingerprint and the sample containing the information to determine a full DNA profile of the person, who stamped his finger in the third area.

32. The system of claim 31, wherein the back side of said laminar cellulose support comprises an adhered label with printings or that bears printings on it.

33. The system of claim 32, wherein the printings are chosen from inscriptions, trademarks, logos, details that enable the provision of instructions for use, batch number, and combinations thereof.

34. The system of claim 31, wherein the graphite area is powder in solid suspension.

35. The system of claim 34, wherein the graphite powder is chosen from activated charcoal in powder, soot, fine graphite powder or super fine graphite powder of any charcoal in solid suspension.

36. The system of claim 35, wherein the graphite solid suspension comprises a material chosen from silicon, polyethylenglycol, high molecular weight fat, synthetic resins, synthetic polymers, or mixes thereof, being placed on a cellulose support such as paper, in such a way that the adherence of the graphite to the support allows its removal when the fingertips are pressed on the surface.

37. The system of claim 31, wherein the area with abrasive is composed of an abrasive surface formed by abrasive paper comprising an abrasive material adhered to a cellulose support, fabric or made of fiber, forming a resinous matrix with an appropriate adhesive.

38. The system of claim 37, wherein the cellulose support is a Kraft paper.

39. The system of claim 37, wherein the abrasive material is selected from Silicon carbide, aluminum oxide, granate, talcum, or mixes thereof.

40. The system of claim 37, wherein the adhesive is of acrylic type.

41. The system of claim 31, wherein the area with abrasive is an abrasive film with a grain size distribution among 100 and 600.

42. The system of claim 31, wherein the third and fourth areas is a pressure sensitive adhesive (PSA).

43. The system of claim 42, wherein the PSA is chosen from adhesives based on rubber, polymer adhesive or copolymer acrylic in solvent base, and polymer adhesive or copolymer acrylic in emulsion.

44. The system of claim 31, wherein the layers of the first, second and third areas are from a polymer selected out of the group of: polyethylene (PE), polypropylene (PP), polystyrene (PS), Polyvinyl chloride (PVC) and polyurethane (PU).

45. The system of claim 44, wherein the polymer has a low, medium or high density and a white, transparent or crystal-like appearance.

46. The system of claim 44, wherein the covers of the first, second and third areas are transparent.

47. The system of claim 44, wherein one of the purposes of the covers of the first, second and third areas is to preserve and maintain the fingerprint and DNA sample obtained free of contamination.

48. The system of any of the claims 31, wherein the system is preserved in the interior of protective wrapper or bag made of wrapper (34) or an ad hoc bag, e.g. made of cellophane or of a plastic material, as polyethylene (PE).
